(19) United States
(12) Patent Application Publication
(10) Pub. No.: US 2026/0263362 A1
(43) Pub. Date: Sep. 10, 2026

(54) STEM CELL-DERIVED ECTO-LIPOSOME FUSION NANOPARTICLES AND USE THEREOF

(71) Applicants: ECTOSOME CO., LTD., Incheon (KR); GACHON UNIVERSITY OF INDUSTRY-ACADEMIC COOPERATION FOUNDATION, Seongnam-si (KR)

(72) Inventors: Dongwoo KHANG, Seongnam-si (KR); Junyoung Park, Seongnam-si (KR); Geun Hye Kim, Seongnam-si (KR); In Ah Kwon, Seongnam-si (KR)

(21) Appl. No.: 19/671,649

(22) Filed: May 8, 2026

Related U.S. Application Data
(63) Continuation of application No. PCT/KR2024/015123, filed on Oct. 4, 2024.

(30) Foreign Application Priority Data
Nov. 9, 2023 (KR) 10-2023-0154426

(51) Int. Cl. A61K 9/127; A61K 31/175; A61K 31/196; A61K 31/255; A61K 31/282; A61K 31/337; A61K 31/4745; A61K 31/475; A61K 31/675; A61K 33/243; A61P 35/00

(52) U.S. Cl. CPC A61K 9/127; A61K 31/175; A61K 31/196; A61K 31/255; A61K 31/282; A61K 31/337; A61K 31/4745; A61K 31/475; A61K 31/675; A61K 33/243; A61P 35/00

(57) ABSTRACT

The present invention relates to novel ecto-liposome fusion nanoparticles, and more specifically, provides novel stem cell-derived ecto-liposome fusion nanoparticles exhibiting superior targeting capability to a tumor site compared to conventional drug delivery carriers having targeting capability.

### CROSS-REFERENCE TO RELATED APPLICATIONS

This application is a bypass continuation of international application no. PCT/KR2024/015123, filed on Oct. 4, 2024, which claims priority to and the benefit of Korean Patent Application No. 10-2023-0154426, filed on Nov. 9, 2023, the disclosures of which are incorporated herein by reference in their entireties.

### TECHNICAL FIELD

The present invention relates to novel ecto-liposome fusion nanoparticles, and more particularly, to stem cell-derived ecto-liposome fusion nanoparticles having enhanced targeting capability to a tumor site.

### BACKGROUND ART

Ectosomes are vesicles of various sizes (diameter: 0.1 to 1 mm) that directly bud from the plasma membrane and are secreted into the extracellular space. Unlike living cells, ectosomes have phosphatidylserine, a phospholipid, on their surface. Although ectosomes are generated and secreted by direct budding of the cell membrane, it is known that the cargo components contained therein are not identical to those of the parent cell and vary depending on the state of the cell (Cocucci and Meldolesi, Curr. Biol. 21 (23): R940-R940, 2011). Ectosomes have different biogenesis mechanism and substances contained therein from exosomes, which are secreted to the outside of the cell via the multivesicular body (MVB) pathway from; however, it is known that their interactions in the extracellular space and their mode of interaction with target cells are similar to those of exosomes. However, while exosomes have received considerable attention in functional aspects such as disease treatment and diagnosis, ectosomes have been relatively less studied.

Meanwhile, liposomes are spherical vesicular structures composed of a lipid bilayer, consisting of phospholipids, which may be artificially prepared or found in cells. Having both hydrophobic and hydrophilic properties, they can selectively absorb substances from the surrounding environment through the membrane and increase in size. Liposomes are mostly spherical or ellipsoidal, and since there is an internal space therein, drugs such as anticancer agents can be encapsulated and delivered into the body, thereby being utilized as drug delivery carriers. In this regard, Korean Patent Laid-Open Publication No. 2021-0063254 discloses a composition for assisting anticancer therapy comprising a liposome composition and a method for drug delivery using the same.

### DETAILED DESCRIPTION OF THE INVENTION

### Technical Problem

However, in the case of the above-described prior art, since it relates to an immunotherapy that treats cancer by disabling the cancer's ability to evade immune cells, there is a problem in that intracellular delivery efficiency is low.

The present invention has been made to solve various problems including the above-described problems, and an object thereof is to provide a novel stem cell-derived ecto-liposome fusion nanoparticle exhibiting superior targeting capability to a tumor site compared to conventional drug delivery carriers having targeting capability. However, this object is illustrative, and the scope of the present invention is not limited thereby.

### Technical Solution

According to one aspect of the present invention, there is provided a fusion nanoparticle formed by fusion of a stem cell-derived ectosome and a liposome.

According to another aspect of the present invention, there is provided a drug delivery carrier comprising the fusion nanoparticle as an active ingredient.

According to another aspect of the present invention, there is provided a pharmaceutical composition comprising the drug delivery carrier and an active drug.

According to another aspect of the present invention, there is provided a pharmaceutical composition for treating cancer, comprising as an active ingredient a fusion nanoparticle formed by fusion of a stem cell-derived ectosome and a liposomem, wherein the fusion nanoparticle is loaded with an anticancer drug.

According to another aspect of the present invention, there is provided a method for preparing a fusion nanoparticle having targeting capability to a tumor tissue, comprising
isolating ectosomes from stem cells educated by culturing the stem cells in a conditioned medium of tumor tissue; and
fusing the ectosomes and liposome nanoparticles through utlrasonication after mixing the ectosomes with the liposome nanoparticles.

According to another aspect of the present invention, there is provided a use of a fusion nanoparticle, which is formed by fusion of a stem cell-derived ectosome and a liposome, wherein the nanoparticle is loaded with an anticancer drug, for the manufacture of a medicament for preventing and treating cancer.

According to another aspect of the present invention, there is provided a method for treating cancer in a subject, the method comprising administering to the subject in need thereof a therapeutically effective amount of a fusion nanoparticle formed by fusion of a stem cell-derived ectosome and a liposome, wherein the fusion nanoparticle is loaded with an anticancer drug.

### Effect of the Invention

As described above, the stem cell-derived ecto-liposome fusion nanoparticle of the present invention exhibits enhanced targeting capability to a target disease site, namely a tumor site. Thus, it may be utilized as a drug delivery carrier capable of releasing a drug, such as an anticancer agent, systemically or locally by loading the drug therein, and may be used as a therapeutic agent for the treatment of intractable cancer or metastatic tumors. However, the scope of the present invention is not limited by these effects.

### BRIEF DESCRIPTION OF THE DRAWINGS

FIG. 1 is a schematic diagram schematically illustrating a preparation process of ecto-liposome fusion nanoparticles in which adipose-derived mesenchymal stem cell (AD-MSC)-derived ectosomes and liposomes are fused according to the present invention.

FIG. 2 is a transmission electron microscopy (TEM) image showing results of observing whether ectosomes and liposomes are fused after preparation of the AD-MSC-derived ecto-liposome fusion nanoparticles according to the present invention, and a graph showing analysis results thereof.

FIG. 3 is a graph showing results of analyzing hydrodynamic sizes and electrical potentials of liposomes, AD-MSC-derived ectosomes, and ecto-liposome fusion nanoparticles.

FIG. 4 is a graph showing results of analyzing a size distribution of the ecto-liposome fusion nanoparticles according to the present invention by nanoparticle tracking analysis (NTA).

FIG. 5 is a gel image showing results of confirming whether liposomes, ectosomes, and ecto-liposome fusion nanoparticles are fused by sodium dodecyl sulfate-polyacrylamide gel electrophoresis (SDS-PAGE).

FIG. 6a is a schematic diagram schematically illustrating a principle of confirming fusion of the ecto-liposome fusion nanoparticles according to the present invention through fluorescence resonance energy transfer (FRET) analysis.

FIG. 6b is a graph showing results of analyzing fluorescence intensity of the ecto-liposome fusion nanoparticles according to the present invention. The ectosomes were labeled with DiI and DiD fluorophores, and analyzed by a FRET experimental method using a Victor 1420 Multilabel Counter (PerkinElmer, Waltham, MA, USA) at an excitation wavelength of 549 nm and an emission wavelength of 665 nm.

FIG. 7 is a fluorescence microscopy image showing targeting capability of the ecto-liposome fusion nanoparticles according to the present invention toward lung cancer cells (A549). In order to evaluate cancer targeting capability of the ecto-liposome fusion nanoparticles, uptake of liposomes stained with a DiO fluorescent material, ectosomes extracted from adipose-derived mesenchymal stem cells (AD-MSC) stained with a DiI fluorescent material, and ecto-liposome fusion nanoparticles stained with DiO and DiI fluorescent materials was visualized by fluorescence.

FIG. 8a shows targeting capability of the ecto-liposome fusion nanoparticles according to the present invention, wherein an upper panel shows a fluorescence image observed using a brain tumor animal model. A xenograft tumor animal model was prepared by injecting brain cancer cells (U87MG) into a brain of an experimental mouse, and a tumor size of the animal was imaged by in vivo fluorescence imaging. A lower panel shows a fluorescence image observing distribution of the ecto-liposome fusion nanoparticles of the present invention in tumor tissue excised from the brain tumor animal model. Distribution of a tumor signal (upper panel) and a drug signal of the ecto-liposome fusion nanoparticles (lower panel) in a brain tumor excised from the experimental animal model was imaged by in vivo fluorescence imaging.

FIG. 8b shows targeting capability of the ecto-liposome fusion nanoparticles according to the present invention, wherein an upper panel shows a fluorescence image observed using a lung tumor animal model. A xenograft tumor animal model was prepared by injecting lung cancer cells (A549) into lungs of an experimental mouse, and a tumor size of the animal was imaged by in vivo fluorescence imaging. A lower panel shows a fluorescence image observing distribution of the ecto-liposome fusion nanoparticles of the present invention in tumor tissue excised from the lung tumor animal model. Distribution of a tumor signal (upper panel) and a drug signal of the ecto-liposome fusion nanoparticles (lower panel) in a lung tumor excised from the experimental animal model was imaged by in vivo fluorescence imaging.

FIG. 9 shows the experimental results for confirming the presence of residual cancer cells during the manufacturing process of AD-MSC-derived ectosomes according to one embodiment of the present invention.

FIG. 10 shows the results of FACS analysis to confirm whether tumor-derived extracellular vesicles (Cancer EVs) are lost by the process of the method for manufacturing ectosome-liposome fusion nanoparticles according to one embodiment of the present invention.

### BEST MODE FOR CARRYING OUT THE INVENTION

### Definitions of Terms in Use

The term “ectosome” used herein refers to a nanosized vesicle having a phospholipid membrane structure with a diameter of 100 to 500 nm, which is generated by budding of the plasma membrane, unlike exosomes among extracellular vesicles.

The term “liposome” used herein refers to a spherical or ellipsoidal vesicle structure formed of lipids. As a structural entity, molecules such as phospholipids possess, within a single molecule, both a hydrophobic portion that repels water and a hydrophilic portion that attracts water. Since liposomes can encapsulate drugs such as anticancer agents therein or reconstitute membrane proteins within the membrane like a biological membrane to reproduce various functions, they have been developed as microcapsules for use as drug delivery carriers in vivo.

The term “drug delivery carrier” used herein refers to a material used to effectively deliver a drug to a target lesion and maintain the drug for an appropriate period of time, and a method of effectively delivering a drug to a lesion using the drug delivery carrier is referred to as a drug delivery system.

### DETAILED DESCRIPTIONS OF THE INVENTION

According to one aspect of the present invention, there is provided a fusion nanoparticle formed by fusion of a stem cell-derived ectosome and a liposome.

In the fusion nanoparticle, the stem cell may be an embryonic stem cell, a mesenchymal stem cell, or an induced pluripotent stem cell, and the mesenchymal stem cell may be a bone marrow-derived stem cell, an umbilical cord blood-derived stem cell, an adipose-derived stem cell, a dental pulp-derived stem cell, or a peripheral blood-derived stem cell.

In the fusion nanoparticle, the stem cell may be a stem cell educated with lesion-derived cells, the lesion may be a tumor tissue, and the tumor tissue may be primary or metastatic.

In the fusion nanoparticle, the educated stem cell may be a stem cell cultured in contact with a culture medium in which the lesion-derived cells are being cultured, and may have a diameter of 100 to 350 nm, and the ectosome and the liposome may be prepared by mixing at a mass ratio of 1:20. Preferably, the ectosome and the liposome may be prepared by mixing at a mass ratio of 1:10.

According to another aspect of the present invention, there is provided a drug delivery carrier comprising the fusion nanoparticle as an active ingredient.

According to another aspect of the present invention, there is provided a pharmaceutical composition comprising the drug delivery carrier and an active drug.

In the pharmaceutical composition, the active drug may be an anticancer drug. In this case, the anticancer drug may be selected from the group consisting of mechlorethamine, chlorambucil, phenylalanine, mustard, cyclophosphamide, ifosfamide, carmustine (BCNU), lomustine (CCNU), streptozotocin, busulfan, thiotepa, cisplatin, carboplatin, doxorubicin, daunorubicin, idarubicin, mitoxantrone, plicamycin, mitomycin, C bleomycin, vincristine, vinblastine, paclitaxel, docetaxel, etoposide, teniposide, topotecan, and irinotecan.

The anticancer drug may be more specifically described as follows:
(i) asparaginase;
(ii) methotrexate;
(iii) pyrimidine analogs, for example, 5-fluorouracil, gemcitabine, and arabinosylcytosine;
(iv) hydroxyurea;
(v) purine analogs, for example, mercaptopurine and thioguanine;
(vi) alkylating agents, for example, nitrogen mustard and cyclophosphamide;
(vii) anthracyclines, for example, anthracycline, doxorubicin, daunorubicin, idarubicin, mitoxantrone, and actinomycin D;
(viii) mitotic inhibitors, for example, vincristine and taxol;
(ix) anti-angiogenic agents, for example, an antibody specific to vascular endothelial growth factor (VEGF), combretastatin A4, fumagillin, herbimycin A, 2-methoxyestradiol, OGT 2115, TNP 470, tranilast, XRP44X, thalidomide, endostatin, salmosin, angiostatin, or a kringle domain of plasminogen or apolipoprotein;
(x) intercalating agents, for example, carboplatin and cisplatin; and
(xi) radionuclides, for example, ¹⁸F, ⁹⁰Y, ¹⁸⁸Re, ³²P, ⁸⁹Sr, ¹⁶⁵Dy, ¹⁸⁶Re, ¹⁹⁸ Au, ¹⁵³Sm, ¹³¹I, ¹⁶⁹Er, ¹²⁵I, ⁹⁹Tc, and ¹⁶⁶Ho.

According to another aspect of the present invention, there is provided a pharmaceutical composition for treating cancer, comprising, as an active ingredient, a fusion nanoparticle formed by fusion of a stem cell-derived ectosome and a liposome, wherein the fusion nanoparticle is loaded with an anticancer drug.

In the pharmaceutical composition, the stem cell may be an educated stem cell educated with cancer cells to be treated, and the anticancer drug may be loaded by being bound to a surface or interior of the fusion nanoparticle via covalent or non-covalent bonding, or by being encapsulated within a nanoparticle having a core-shell structure. More specifically, the anticancer drug may be loaded into the liposome nanoparticle by being bound to a surface or interior of the nanoparticle via covalent or non-covalent bonding, or by being encapsulated within a nanoparticle having a core-shell structure, and when loaded via covalent bonding, the anticancer agent may be covalently bound to a terminal or side chain of a biodegradable polymer. Optionally, the anticancer agent may also be loaded by being bound to a surface of the ectosome membrane via a covalent or non-covalent bonding method and presented on the surface thereof.

In the pharmaceutical composition, the cancer may be selected from the group consisting of brain tumor, lung cancer, gastric cancer, liver cancer, bone cancer, pancreatic cancer, gallbladder cancer, bile duct cancer, skin cancer, head and neck cancer, melanoma, uterine cancer, ovarian cancer, rectal cancer, colorectal cancer, colon cancer, breast cancer, uterine sarcoma, esophageal cancer, laryngeal cancer, small intestine cancer, and thyroid cancer, but is not limited thereto.

According to another aspect of the present invention, there is provided a method for treating cancer in a subject, comprising administering to the subject in need thereof a fusion nanoparticle formed by fusion of a stem cell-derived ectosome and a liposome.

According to another aspect of the present invention, there is provided a method for preparing a fusion nanoparticle having targeting capability to a tumor tissue, comprising
isolating ectosomes from stem cells educated by culturing the stem cells in a conditioned medium of tumor tissue; and
fusing the ectosomes and liposome nanoparticles through utlrasonication after mixing the ectosomes with the liposome nanoparticles.

In the method, the condition medium may be free of tumor cells, and for this purpose, the condition medium may be prepared by filtering a tumor tissue culture medium using a 100 to 300 nm filter.

In the method, the ectosome-liposome fusion nanoparticle generated through the cryogenic freeze-thaw process may be homogenized through an extrusion process. The cryogenic freeze-thaw process may be repeated 2 to 5 times. The extrusion process may be performed using an extruder with a 0.2 to 2 μm filter membrane and can be repeated 5 to 20 times.

According to another aspect of the present invention, there is provided a use of a fusion nanoparticle, which is formed by fusion of a stem cell-derived ectosome and a liposome, wherein the fusion nanoparticle is loaded with an anticancer drug, for the manufacture of a medicament for preventing and treating cancer.

According to another aspect of the present invention, there is provided a method for treating cancer in a subject, the method comprising administering to the subject in need thereof a therapeutically effective amount of a fusion nanoparticle formed by fusion of a stem cell-derived ectosome and a liposome, wherein the fusion nanoparticle is loaded with an anticancer drug.

The pharmaceutical composition of the present invention may vary depending on the type of affected site of a patient, an application site, a number of administrations, an administration time, a formulation, a condition of the patient, a type of additive, and the like. Although the dosage is not particularly limited, it may be 0.01 μg/kg/day to 10 mg/kg/day. The daily dose may be administered once per day, or divided into two to three administrations per day at appropriate intervals, or may be intermittently administered at intervals of several days.

In the pharmaceutical composition of the present invention, the compound may be administered orally or parenterally, and preferably may be administered parenterally by intravenous injection, subcutaneous injection, intracerebroventricular injection, intracerebrospinal fluid injection, intramuscular injection, or intraperitoneal injection.

The pharmaceutical composition of the present invention may further comprise suitable carriers, excipients, and diluents commonly used in the preparation of pharmaceutical compositions. In addition, solid or liquid formulation additives may be used in the preparation of the pharmaceutical composition. The formulation additives may be either organic or inorganic. Examples of excipients include lactose, sucrose, white sugar, glucose, cornstarch, starch, talc, sorbitol, crystalline cellulose, dextrin, kaolin, calcium carbonate, and silicon dioxide. Examples of binders include polyvinyl alcohol, polyvinyl ether, ethyl cellulose, methyl cellulose, gum arabic, tragacanth, gelatin, shellac, hydroxypropyl cellulose, hydroxypropyl methylcellulose, calcium citrate, dextrin, and pectin. Examples of lubricants include magnesium stearate, talc, polyethylene glycol, silica, and hydrogenated vegetable oil. As a coloring agent, any coloring agent permitted for addition to pharmaceuticals may be used. Tablets and granules thereof may be coated with sugar coating, gelatin coating, or other suitable coatings as necessary. In addition, preservatives, antioxidants, and the like may be added as needed. Furthermore, when the pharmaceutical composition is a drug, it may further comprise one or more selected from fillers, anti-caking agents, lubricants, wetting agents, flavoring agents, emulsifying agents, and preservatives. Meanwhile, the formulation of the pharmaceutical composition of the present invention may be in a preferred form depending on the method of use, and in particular, it is preferable to formulate the composition by adopting methods known in the art so as to provide rapid, sustained, or delayed release of an active ingredient after administration to a mammal. Specific examples of formulations include plasters, granules, lotions, liniments, lemonades, powders, syrups, liquids and solutions, aerosols, extracts, elixirs, fluid extracts, emulsions, suspensions, decoctions, infusions, tablets, suppositories, injections, spirits, cataplasms, capsules, troches, tinctures, pastes, pills, and soft or hard gelatin capsules.

The pharmaceutical composition of the present invention may further comprise components commonly used in pharmaceutical compositions, for example, conventional adjuvants such as stabilizers, solubilizers, and flavoring agents, as well as carriers.

The pharmaceutical composition or an intra-articular injection formulation may comprise various carriers suitable for direct injection into an affected site.

Pharmaceutically acceptable carriers included in the pharmaceutical composition of the present invention are those commonly used in formulation, and include, but are not limited to, lactose, dextrose, sucrose, sorbitol, mannitol, starch, gum acacia, calcium phosphate, alginate, gelatin, calcium silicate, microcrystalline cellulose, polyvinylpyrrolidone, cellulose, water, syrup, methyl cellulose, methylhydroxybenzoate, propylhydroxybenzoate, talc, magnesium stearate, and mineral oil. The pharmaceutical composition of the present invention may further comprise, in addition to the above components, lubricants, wetting agents, sweeteners, flavoring agents, emulsifying agents, suspending agents, preservatives, and the like. Suitable pharmaceutically acceptable carriers and formulations are described in detail in Remington's Pharmaceutical Sciences (19th Ed., 1995).

The present inventors have previously found, through prior studies, that stem cells educated with cancer cell-conditioned medium or inflammatory cell-conditioned medium exhibit improved targeting capability to tumor tissues or inflammatory tissues without the use of unnecessary processes such as genetic manipulation (Korean Patent Laid-Open Publication No. 2022-0117474). Thereafter, the present inventors hypothesized that ectosomes secreted from the educated stem cells would also have targeting capability to lesions such as tumor tissues, and in order to verify the hypothesis, ectosomes were isolated from the educated stem cells and then mixed with biodegradable polymer nanoparticles, specifically poly (lactic-co-glycolic acid) (PLGA) nanoparticles, to prepare ectosome-biodegradable fusion nanoparticles coated with an ectosome membrane. As a result of investigating whether the fusion nanoparticles are effectively targeted to tumor tissues, it was confirmed that the fusion nanoparticles exhibited significantly higher tumor cell targeting capability compared to ectosome-biodegradable polymer fusion nanoparticles derived from non-educated stem cells, and that the fusion nanoparticles exhibited further enhanced targeting capability to tumor tissues.

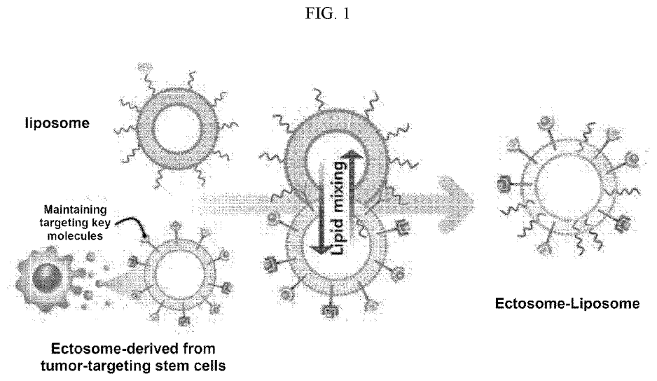

Furthermore, the present inventors prepared novel fusion nanoparticles by isolating ectosomes from the educated stem cells and fusing the same with liposomes, which are spherical vesicular structures composed of an artificially prepared lipid bilayer (see FIG. 1). Thereafter, as a result of analyzing tumor tissue targeting capability of the ecto-liposome fusion nanoparticles of the present invention using lung cancer and brain tumor animal models, it was confirmed that the ecto-liposome fusion nanoparticles derived from stem cells educated with cancer cell-conditioned medium exhibited excellent targeting capability to brain tumor and lung cancer tissues. These results provide a basis that the ecto-liposome fusion nanoparticles of the present invention can overcome a technical limitation in which anticancer drug treatment has been restricted due to the presence of the blood-brain barrier, and suggest that the nanoparticles may be effectively used as a drug delivery carrier for delivering active drugs to lesions by increasing targeting capability to lesions in intractable cancers such as lung cancer.

Hereinafter, the present invention will be described in more detail with reference to Examples. However, the present invention is not limited to the Examples disclosed below, but may be implemented in various different forms, and the following Examples are provided to make the disclosure of the present invention complete and to fully convey the scope of the invention to those skilled in the art.

### Example 1: Isolation of Adipose-Derived Mesenchymal Stem Cell (AD-MSC)-Derived Ectosomes

The present inventors isolated ectosomes (nanovesicles) in order to prepare fusion nanoparticles in which adipose-derived mesenchymal stem cell (AD-MSC)-derived ectosomes (nanovesicles) having enhanced targeting capability by education with cancer cells are fused with liposomes.

### 1-1: Method for Educating Stem Cells

In order to enhance targeting capability to lung cancer cells, adipose-derived mesenchymal stem cells (AD-MSC) and lung cancer cells (A549) were seeded at 5×10⁵ cells per well in a 100 culture plate and cultured at 37° C. for 24 hours. In addition, in order to enhance targeting capability to brain cancer cells, adipose-derived mesenchymal stem cells (AD-MSC) and brain cancer cells (U87MG) were seeded at 5×10⁵ cells per well in a 100π culture plate and cultured at 37° C. for 24 hours. Thereafter, a culture medium was obtained from a plate in which the lung cancer cells were being cultured by collecting cancer cell culture medium in a 50 ml conical tube, centrifuging at 1,500 rpm, and then filtering through 200 nm filter (Syringe Filter Minisart®, Satorius Inc.), and 10 mL of the conditional medium was added to the stem cells followed by additional culture for 24 hours (education).

### 1-2: Isolation of Ectosomes from Educated Stem Cells

To a plate in which adipose-derived mesenchymal stem cells (AD-MSC) educated with the culture medium of the lung cancer cells or the brain cancer cells were cultured, 5 μL of cytochalasin B (10 μg/μL) was added, and the cells were detached and vortexed for 3 minutes. Subsequently, the resulting mixture was centrifuged at 1,000 rpm for 10 minutes to collect a supernatant, and then centrifuged at 4,000 rpm for 15 minutes to remove the supernatant, thereby obtaining an ectosome pellet. The pellet was resuspended in 10 μL of distilled water (DW) and quantified using a nanodrop.

### Example 2: Ecto-Liposome Fusion

### 2-1: Preparation of Liposome Nanoparticles

Since liposomes are composed of phospholipids and thus have biocompatibility as drug delivery carriers, the present inventors synthesized liposomes using microfluidic technology. Specifically, in a lipid phase, 18:1 (Δ9-Cis)PC (DOPC) [CAS #: 850375P, Avanti], cholesterol [CAS #: C8667, Sigma], and 18:0 PEG2000 PE [CAS #: 880120, Avanti] were added to ethanol at a molar ratio of 60:35:5, followed by vortexing and sonication for 10 minutes to prepare the lipid phase. Subsequently, triple-distilled water was prepared as an aqueous phase, and synthesis was performed using a microfluidic chip having a pore size of 5 μm at flow rates of 8 mL/min for the aqueous phase and 0.8 mL/min for the lipid phase. Thereafter, residual liposomes that were not nanoparticulated were removed by centrifugation at 4° C. and 3000×g for 20 minutes using a 100 kDa filter (Merck, Amicon UFC910024 Amicon® Ultra-15 Centrifugal Filter Unit, 100K).

### 2-2: Preparation of Ectosome-Fused Liposome Nanoparticles

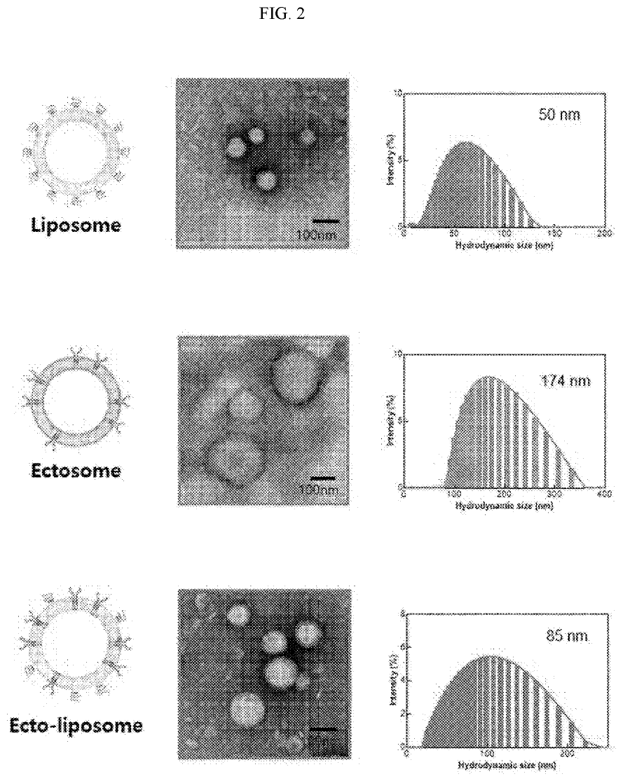

The ectosomes extracted according to Example 1 and the liposome nanoparticles prepared according to Example 2-1 were mixed at a mass ratio of 1:10, and subjected to ultrasonication in a bath sonicator for 10 minutes. Thereafter, a cycle consisting of 10 minutes freezing in liquid nitrogen and 10 minutes of thawing at 37° C. was repeated for a total of three cycles for the fusion. Subsequently, the mixture extruded by extrusion using an Avanti Mini-Extruder (Avanti Polar Lipids, USA). The extrusion was repeated 10 times through a 1.0 μm polycarbonate membrane to generate the final hybrid nanoparticles with a uniform size distribution. Subsequently, residual ecto-liposome fusion nanoparticles that were not properly nanoparticulated were removed by centrifugation at 4° C. and 3000×g for 20 minutes using a 100 kDa filter (Merck, Amicon UFC910024 Amicon® Ultra-15 Centrifugal Filter Unit, 100K), and whether the liposomes and ectosomes were properly fused was confirmed using transmission electron microscopy (TEM) and a FRET assay (FIG. 2).

### Example 3: Preparation of DiD-, DiI-, and DiO-Labeled Ecto-Liposomes

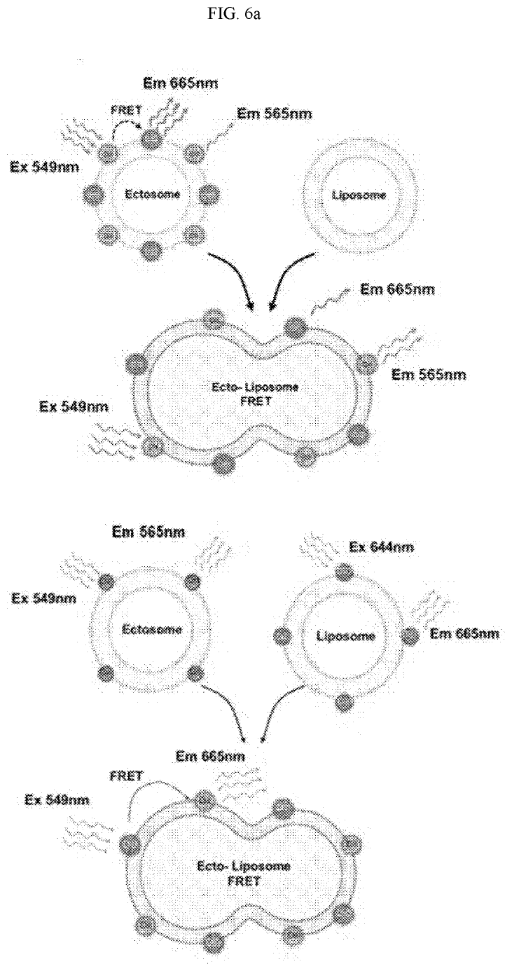

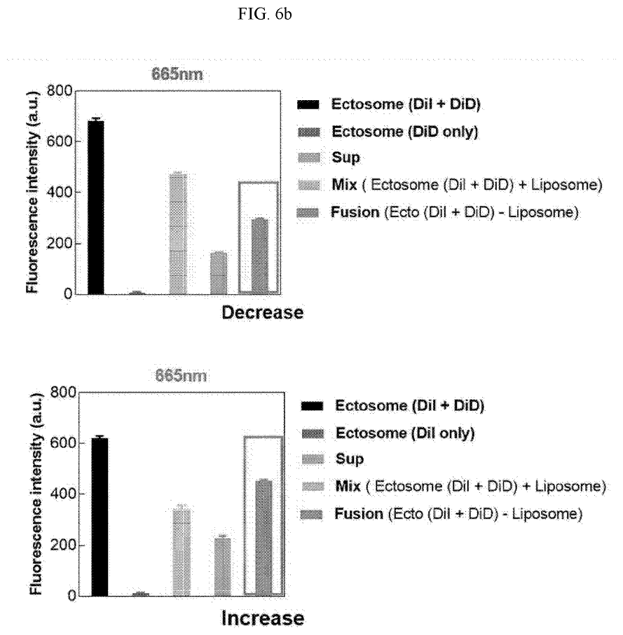

In order to prepare a fusion construct in which a fluorescent dye DiI is attached to ectosomes, the present inventors treated DiI at 5 μL/1 mL prior to Example 1-2 and incubated for 15 minutes. Thereafter, in order to attach fluorescent dyes DiD or DiO to liposomes, liposomes were synthesized as in Example 2-1, followed by treatment with DiD or DiO at 5 μL/1 mL and vortexing and sonication for 10 minutes. Subsequently, residual liposomes were removed by centrifugation at 4° C. and 3000×g for 20 minutes using a 100 kDa filter (Merck, Amicon UFC910024 Amicon® Ultra-15 Centrifugal Filter Unit, 100K). Thereafter, fusion was confirmed using a FRET assay and an uptake assay (FIG. 6a and FIG. 6b).

### Example 4: Sodium Dodecyl Sulfate-Polyacrylamide Gel Electrophoresis (SDS-PAGE)

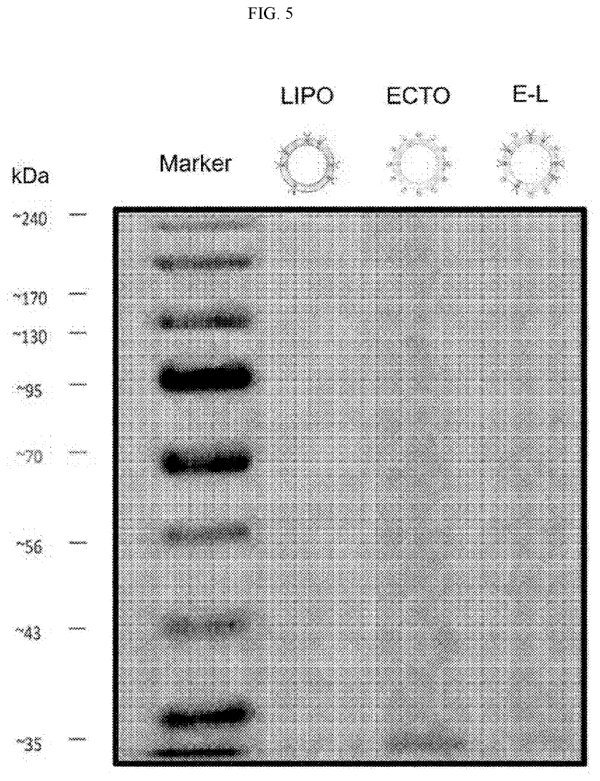

In order to confirm fusion of the ectosome-liposome fusion nanoparticles prepared above, the present inventors performed SDS-PAGE. Specifically, proteins present in the ectosome-liposome fusion nanoparticles were separated and purified by SDS-PAGE, and each of the liposomes, ectosomes, and ecto-liposomes was mixed with a lysis buffer and incubated on ice for 30 minutes. Thereafter, centrifugation was performed at 4° C. and 3000×g for 20 minutes to obtain a supernatant, and proteins were quantified by a Coomassie assay. Subsequently, 30 μg of each of the liposomes, ectosomes, and ecto-liposomes was mixed with 4X SDS sample buffer, incubated at 95° C. for 5 minutes, and then incubated on ice again for 5 minutes. Then, centrifugation was performed at 4° C. and 13,000 rpm for 5 minutes to obtain a supernatant, and electrophoresis (SDS-PAGE) was performed by loading onto a gel at 5 mV for 20 minutes. Thereafter, the gel was stained with Coomassie blue for 1 hour, and destaining was performed for 30 minutes to remove residual staining reagent, followed by scanning to confirm protein bands. As a result, protein bands were not observed in the liposomes, whereas similar protein band patterns were observed in the ectosomes and the ecto-liposomes, thereby confirming ecto-liposome fusion (FIG. 5).

### Experimental Example 1: Physicochemical Properties

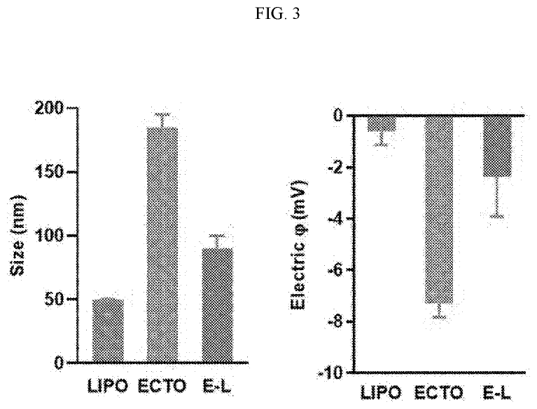

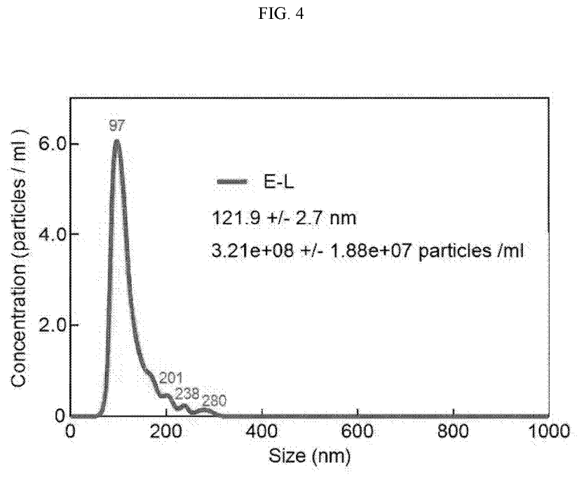

The size distribution and zeta potential of the ecto-liposome fusion nanoparticles of the present invention were analyzed using a particle size analyzer (Anton Paar LiteSizer 500), and nanoparticle tracking analysis (NTA) was performed using a Nanosight NS300 (Nanosight, UK). As a result, as shown in FIG. 3 and FIG. 4, the liposomes exhibited an average size of 50 nm, the ectosomes exhibited an average size of 184 nm, and the ecto-liposome fusion nanoparticles (ecto-liposome) exhibited an average size of 90 nm. In addition, the liposomes exhibited a charge of −1 mV, the ectosomes exhibited a charge of −7.3 mV, and the ecto-liposome fusion nanoparticles exhibited a charge of −2.38 mV. Through the NTA analysis, it was confirmed that the ecto-liposome fusion nanoparticles have a size of 121.9±2.7 nm and a particle number of 3.21×10⁸±1.88×10⁷ particles per mL.

### Experimental Example 2: Analysis of Uptake Capability of Ecto-Liposome Fusion Nanoparticles

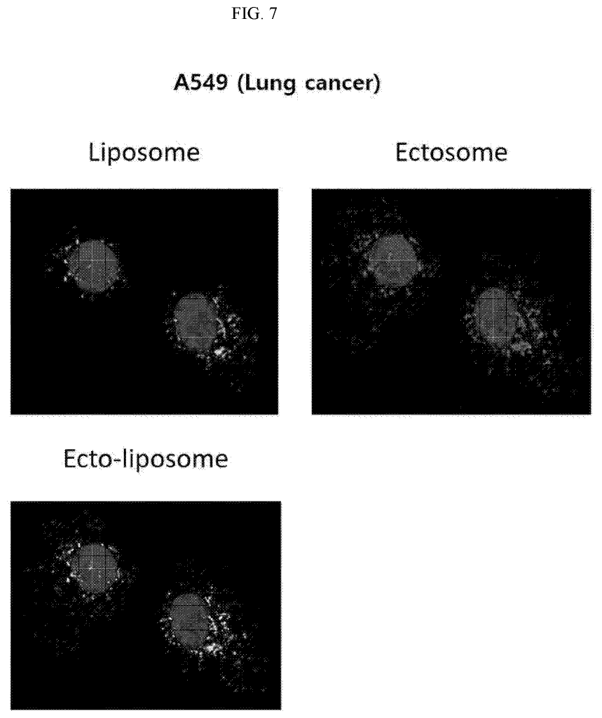

The present inventors investigated uptake capability of the ectosome-liposome fusion nanoparticles of the present invention with respect to tumor cells. Specifically, lung cancer cells (A549) were seeded (5×10⁴ cells) on a poly-D-lysine-coated coverslip and cultured overnight. Thereafter, DiO-labeled liposome nanoparticles, DiI-labeled ectosomes isolated from lung cancer cells, and DiI-labeled ecto-liposome fusion nanoparticles (ecto-liposome) isolated from lung cancer cells were added to the cultured cells and incubated at 37° C. for 6 hours. Thereafter, all samples were fixed overnight with 4% paraformaldehyde in an intermediate medium at 4° C., and DiO and DiI fluorescence within the tumor cells was imaged using an LSM700 confocal microscope (Carl Zeiss). As a result, as shown in FIG. 7, only DiO fluorescence was observed in tumor cells treated with liposome nanoparticles, and only DiI fluorescence was observed in tumor cells treated with ectosomes, whereas both DiO and DiI fluorescence were observed within tumor cells treated with the ecto-liposome fusion nanoparticles.

### Experimental Example 3: Analysis of Tumor Tissue Targeting Capability in a Brain Tumor Model

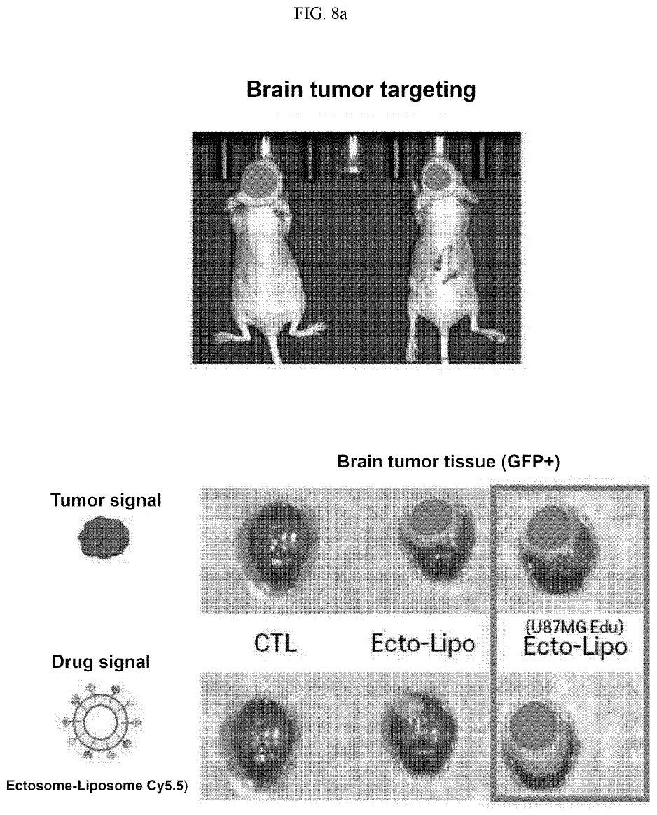

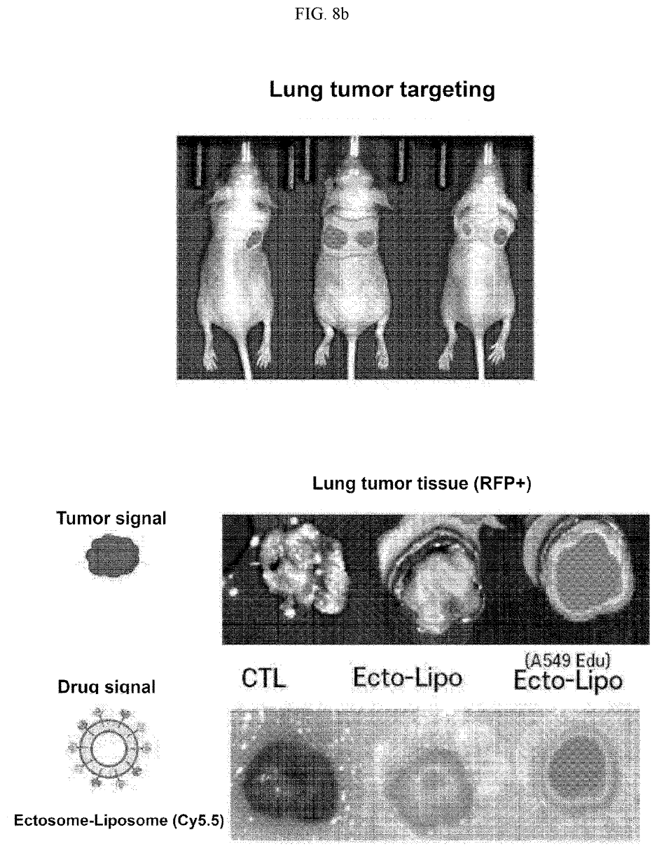

The present inventors analyzed tumor tissue targeting capability of the ecto-liposome fusion nanoparticles of the present invention using a brain tumor animal model. Specifically, the brain tumor animal model was prepared by directly injecting, into the brain of BALB/c nude mice, brain tumor cells genetically engineered to express a fluorescent protein and a bioluminescent enzyme (luciferase) (U87MG-luciferase-GFP, 3×10⁵ cells) via a surgical procedure. Thereafter, tumor size was monitored using an in vivo imaging system (IVIS imaging system), and after confirming formation of a brain tumor, ecto-liposomes (1×10⁶ cells) extracted from educated AD-MSCs, educated AD-MSCs (1×10⁶ cells), and liposomes were intravenously injected once daily for a total of three days via a tail vein of the brain tumor model mice (FIG. 8a). Thereafter, on day 4, whole-body in vivo imaging of the experimental animals was performed, and after sacrificing the animals, brains in which tumors had formed were excised and tumor tissue targeting capability of the ecto-liposomes was analyzed through ex vivo imaging.

As a result, as shown in FIG. 8a, it was confirmed that, in the case of ecto-liposomes educated with a culture medium of brain cancer cells, targeting capability to brain tumor tissue was enhanced. The above results were achieved by intravenously administering stem cells to the experimental animals, demonstrating that the stem cells traverse the blood-brain barrier and accurately migrate to the brain tumor, thereby suggesting that the present invention provides an important basis for overcoming a technical limitation in which anticancer drug treatment has been restricted due to the presence of the blood-brain barrier in brain tumor therapy.

### Experimental Example 4: Analysis of Tumor Tissue Targeting Capability in a Lung Cancer Model

The present inventors also analyzed tumor tissue targeting capability of the ecto-liposome fusion nanoparticles of the present invention using a lung cancer animal model. Specifically, the lung cancer animal model was prepared by injecting, via a tail vein, lung cancer cells genetically engineered to express a fluorescent protein and a bioluminescent enzyme (luciferase) (A549-luciferase-RFP, 1×10⁶ cells) into BALB/c nude mice. Thereafter, tumor growth was monitored for 4 weeks using an in vivo imaging system (IVIS imaging system) (FIG. 8b), and after confirming formation of lung cancer, ecto-liposomes (1×10⁶ cells) extracted from educated AD-MSCs, educated AD-MSCs (1×10⁶ cells), and liposomes were intravenously injected once via a tail vein of the lung cancer model mice. Six hours later, whole-body in vivo imaging of the experimental animals was performed, and after sacrificing the animals, lungs in which tumors had formed were excised and tumor tissue targeting capability of the ecto-liposomes was analyzed through ex vivo imaging.

As a result, as shown in FIG. 8b, it was confirmed that, in the case of ecto-liposomes educated with a culture medium of lung cancer cells, targeting capability to tumor tissue was enhanced. These results suggest that educated stem cells may be very effectively used for cancer cell-specific drug delivery, particularly for drug delivery to lung cancer, which is a representative intractable malignant tumor.

### Experimental Example 5: Identification of Removal of Residual Cancer Cells During Preparing Ectosomes from Educated AD-MSCs

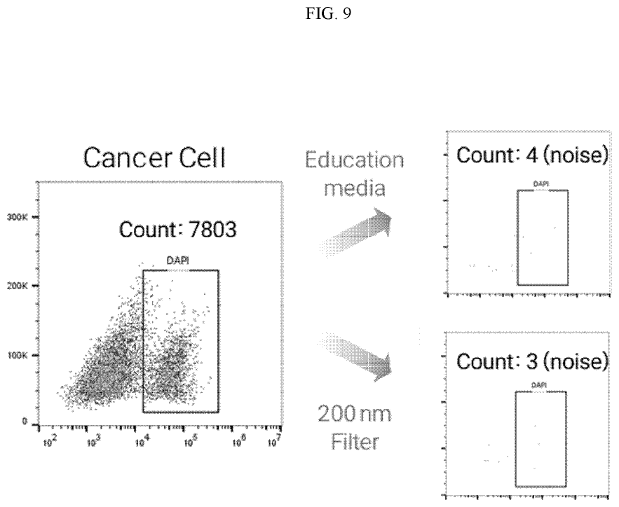

The inventors aimed to determine whether residual cancer cells and cancer-derived materials remained in the final product of the ectosome manufacturing process described in Examples 1 and 2. To this end, the number of cancer cells was quantified using flow cytometry (SA3800, Sony Biotechnology). As shown in FIG. 9, the count of the initial cancer cell population was 7,803; however, this count decreased sharply to 4 (noise level) in the education media used for the education of adipose-derived mesenchymal stem cells (AD-MSCs). Furthermore, following filtration through a 200 nm pore-size filter, the count was 3, confirming that residual cancer cells were substantially absent. This demonstrates that the filtration process of the present invention physically blocks nearly 100% of intact cells capable of inducing tumorigenicity (oncogenic potential).

### Experimental Example 6: Confirmation of Loss of Cancer Cell-Derived Extracellular Vesicles (Cancer EVs) at Each Process Step

The present invention is characterized by a dual-safety process that biochemically removes minute cancer-cell-derived secretory materials, or Cancer EVs (exosomes), via a nanoparticle fusion synthesis step, in addition to physical cell removal. Accordingly, the inventors aimed to verify, through rigorous stress testing, whether the process of the present invention completely eliminates cancer-cell-derived extracellular vesicles (Cancer EVs).

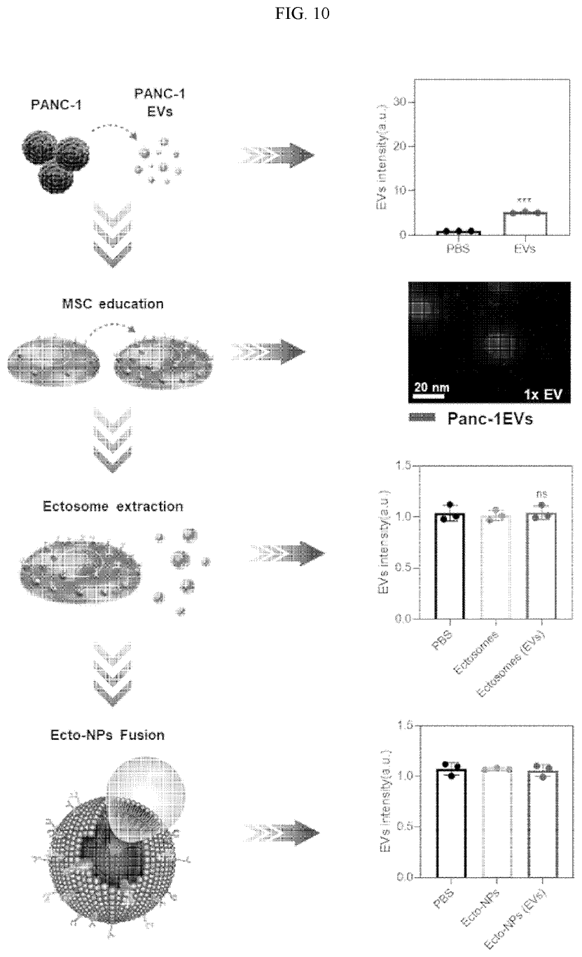

Specifically, the inventors prepared a solution of extracellular vesicles isolated from PANC-1 pancreatic cancer cells, used it to educate AD-MSCs, and quantified the number of cancer-derived EVs at each step of the ectosome-liposome fused nanoparticle manufacturing process using FACS analysis. For the FACS analysis EVs derived from PANC-1 cells were labeled with fluorescence dye using PKH26 Red Fluorescent Cell Linker Mini Kit (Cat #: MINI26). As shown in FIG. 10, FACS analysis results showed that cancer cell-derived EVs were not detected from the stage of training AD-MSCs with conditioned media, and similarly, cancer cell-derived EVs were not detected in the next step, the Ectosome extraction step, nor were cancer cell-derived EVs detected in the Ectosome-Liposome fusion nanoparticle manufacturing process.

In conclusion, the ectosome-liposome fused nanoparticles of the present invention are secured by a dual safety mechanism consisting of: (1) the removal of cancer cells via a 200 nm physical filtration process; and (2) the biochemical dilution and washing of tumor-derived Cancer EVs via the fused nanoparticle synthesis process. Therefore, the drug delivery vehicle and its manufacturing method according to the present invention offer the exceptional effect of providing a highly safe and pure targeted drug delivery vehicle, in which the risks of tumorigenicity and immunogenicity are significantly eliminated upon in vivo administration, despite the use of tumor cell culture media for stem cell education.

Although the present invention has been described with reference to the above-described Experimental Examples, these are merely illustrative, and those skilled in the art will understand that various modifications and equivalent embodiments may be made therefrom. Accordingly, the true technical scope of the present invention should be defined by the technical spirit of the appended claims.

(Government-funded Research and Development Project Supporting the Present Invention)

(Unique Project Number) 2022398018

(Project Number) 2022M3A9G8018189

(Ministry) Ministry of Science and ICT

(Managing Agency) National Research Foundation of Korea

(Research Program) Bio and Medical Technology Development Program (Bio-origin Convergence Technology Development)

(Project Title) Development of Source Technology for Targeting Complex Tumors Using Patient-Specific Ecto-Liposomes

(Contribution Ratio) 1/1

(Performing Institution) Gachon University Industry-Academic Cooperation Foundation

(Project Period) Jan. 1, 2023 to Dec. 31, 2023

1. A fusion nanoparticle formed by fusion of a stem cell-derived ectosome and a liposome.

2. The fusion nanoparticle according to claim 1, wherein the stem cell is an embryonic stem cell, a mesenchymal stem cell, or an induced pluripotent stem cell.

3. The fusion nanoparticle according to claim 2, wherein the mesenchymal stem cell is a bone marrow-derived stem cell, an umbilical cord blood-derived stem cell, an adipose-derived stem cell, a dental pulp-derived stem cell, or a peripheral blood-derived stem cell.

4. The fusion nanoparticle according to claim 1, wherein the stem cell is a stem cell educated with lesion-derived cells.

5. The fusion nanoparticle according to claim 4, wherein the lesion is a tumor tissue.

6. The fusion nanoparticle according to claim 5, wherein the tumor tissue is primary or metastatic.

7. The fusion nanoparticle according to claim 4, wherein the educated stem cell is cultured in contact with a culture medium in which the lesion-derived cells are being cultured.

8. The fusion nanoparticle according to claim 1, having a diameter of 100 to 350 nm.

9. The fusion nanoparticle according to claim 1, wherein the ectosome and the liposome are prepared by mixing at a mass ratio of 1:20.

10. A drug delivery carrier comprising the fusion nanoparticle according to claim 1 as an active ingredient.

11. A pharmaceutical composition comprising the drug delivery carrier according to claim 10 and an active drug.

12. The pharmaceutical composition according to claim 11, wherein the active drug is an anticancer drug.

13. The pharmaceutical composition according to claim 12, wherein the anticancer drug is selected from the group consisting of mechlorethamine, chlorambucil, phenylalanine, mustard, cyclophosphamide, ifosfamide, carmustine (BCNU), lomustine (CCNU), streptozotocin, busulfan, thiotepa, cisplatin, carboplatin, doxorubicin, daunorubicin, idarubicin, mitoxantrone, plicamycin, mitomycin, C bleomycin, vincristine, vinblastine, paclitaxel, docetaxel, etoposide, teniposide, topotecan, and irinotecan.

14. A pharmaceutical composition for treating cancer, comprising, as an active ingredient, a fusion nanoparticle formed by fusion of a stem cell-derived ectosome and a liposome, wherein the fusion nanoparticle is loaded with an anticancer drug.

15. The pharmaceutical composition according to claim 14, wherein the stem cell is an educated stem cell educated with cancer cells to be treated.

16. The pharmaceutical composition according to claim 14, wherein the anticancer drug is loaded by being bound to a surface or interior of the fusion nanoparticle via covalent or non-covalent bonding, or by being encapsulated within a nanoparticle having a core-shell structure.

17. The pharmaceutical composition according to claim 14, wherein the cancer is selected from the group consisting of brain tumor, lung cancer, gastric cancer, liver cancer, bone cancer, pancreatic cancer, gallbladder cancer, bile duct cancer, skin cancer, head and neck cancer, melanoma, uterine cancer, ovarian cancer, rectal cancer, colorectal cancer, colon cancer, breast cancer, uterine sarcoma, esophageal cancer, laryngeal cancer, small intestine cancer, and thyroid cancer.

18. A method for preparing a fusion nanoparticle having targeting capability to a tumor tissue, the method comprising:
isolating ectosomes from stem cells educated by culturing the stem cells in a conditioned medium of tumor tissue; and
fusing the ectosomes and liposome nanoparticles by mixing the ectosomes and liposome nanoparticles, applying ultrasonication, and performing a cryogenic freeze-thaw process.

19. The method according to claim 18, wherein the ectosome and the liposome nanoparticles are mixed at a mass ratio of 1:20.

20. A method for treating cancer in a subject, the method comprising administering to the subject in need thereof a therapeutically effective amount of a fusion nanoparticle formed by fusion of a stem cell-derived ectosome and a liposome, wherein the fusion nanoparticle is loaded with an anticancer drug.
